(19) United States
(12) Patent Application Publication
(10) Pub. No.: US 2024/0374843 A1
(43) Pub. Date: Nov. 14, 2024

(54) CANNULA ASSEMBLY

(71) Applicant: Asklepios Biopharmaceutical, Inc., Research Triangle Park, NC (US)

(72) Inventor: Michael W. O'CALLAGHAN, Research Triangle Park, NC (US)

(21) Appl. No.: 18/557,475

(22) PCT Filed: Apr. 20, 2022
(86) PCT No.: PCT/US2022/025609

§ 371 Date: Oct. 26, 2023

Related U.S. Application Data
(60) Provisional application No. 63/180,955, filed on Apr. 28, 2021.
(60) Provisional application No. 63/202,432, filed on Jun. 10, 2021.

(51) Int. Cl. A61M 5/34

(52) U.S. Cl. CPC A61M 5/343; A61M 5/349; A61M 2202/0007; A61M 2205/0216; A61M 2205/0222; A61M 2205/025; A61M 2205/58; A61M 2207/10

(57) ABSTRACT

A cannula assembly includes a cannula, polymeric support material and a hub. The cannula has a proximal end and a distal end. The polymeric support material substantially surrounds a portion of the cannula at or near the proximal end. The hub is configured to be attached to a syringe. The polymeric support material is located between the cannula and the hub. The cannula, the polymeric support material and the hub are adhesively attached.

### CROSS-REFERENCE TO RELATED APPLICATIONS

This application claims priority to U.S. Provisional Patent Application No. 63/180,955, filed Apr. 28, 2021 and U.S. Provisional Patent Application Ser. No. 63/202,432, filed Jun. 10, 2021, each of which is hereby incorporated by reference in its entirety.

### FIELD OF THE INVENTION

The present invention relates to a cannula assembly and, more particularly, to a cannula assembly including a hub for connecting to a syringe.

### BACKGROUND OF THE INVENTION

Cannulas have been used in combination with syringes to deliver or receive fluid. One problem associated with the use of cannulas and the components used in conjunction with the same is sterilization. The components of the cannula are often sterilized individually and then assembled in a sterile surgical field before being attached to a syringe. Some cannula designs can be susceptible to breakage, as well as having an undesirable amount of dead space where unaccounted liquid can accumulate.

It would be desirable to have a simplified method of forming cannula assemblies that can be sterilized efficiently, as well as having an efficient cannula assembly design that prevents or inhibits breakage of the cannula and reduces dead space when used with syringes.

### SUMMARY OF THE INVENTION

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings and each claim.

According to one aspect of the present disclosure, a cannula assembly comprises a cannula, polymeric support material, and a hub. The cannula has a proximal end and a distal end. The polymeric support material substantially surrounds a portion of the cannula at or near the proximal end. The hub is configured to be attached to a syringe. The polymeric support material is located between the cannula and the hub. The cannula, the polymeric support material and the hub are adhesively attached.

According to a configuration of the above implementation, the cannula comprises glass fibers.

According to another configuration of the above implementation, the polymeric support material is tapered. The polymeric support material is tapered from the proximal end towards the distal end, in which the thickness of the polymeric support material is greater at the proximal end.

According to a further configuration of the above implementation, the polymeric support material comprises polytetrafluoroethylene (PTFE), polyamides, fluoropolymers, polyolefins, PVC (polyvinyl chlorides), polyimides, PEEK (polyetheretherketones), or combinations thereof.

In a further aspect of the above implementation, the polymeric support material is polymeric support tubing.

In a further aspect of the above implementation, the polymeric support material completely surrounds the cannula.

In yet a further aspect of the above implementation, the hub is a Luer-pressure fitting hub. The Luer-pressure fitting hub may include a thread formation configured to attach to the syringe.

In yet a further aspect of the above implementation, the cannula assembly further includes a winged connection adapted to tighten the cannula assembly and the syringe.

In another aspect of the above implementation, the polymeric support material and the hub comprises transparent or translucent material.

In yet a further aspect of the above implementation, the adhesive is a UV-sensitive adhesive.

In another aspect of the above implementation, the cannula assembly further includes overlying tubing in which the overlying tubing is located adjacent to the hub on an opposing side from the polymeric support material.

In yet a further aspect of the above implementation, the polymeric support material is a plurality of polymeric supporting tube segments.

According to a further aspect of the present disclosure, a cannula assembly and a syringe combination includes a cannula and a syringe. The cannula has a proximal end and a distal end. Polymeric support material substantially surrounds a portion of the cannula at or near the proximal end. The polymeric support material is located between the cannula and a hub. The polymeric support material and the hub are adhesively attached. The syringe includes a needle. The hub attaches the cannula and the syringe.

According to a configuration of the above implementation, the polymeric support material is tapered. The polymeric support material is tapered from the proximal end towards the distal end, in which the thickness of the polymeric support material is greater at the proximal end.

According to a further aspect of the present disclosure, the polymeric support material completely surrounds the cannula.

According to a configuration of the above implementation, the hub is a Luer-pressure fitting hub.

According to one method of the present disclosure, a cannula assembly is formed. A cannula is provided having a proximal end and a distal end, polymeric support material, and a hub. The polymeric support material and the hub comprises transparent or translucent material. The polymeric support material is located to substantially surround a portion of the cannula at or near the proximal end. The polymeric support material is located between the cannula and the hub. Adhesive is placed on at least one of the cannula, the polymeric support material, and the hub. The adhesive is exposed to ultra-violet light to securely attach the cannula, the polymeric support material, and the hub.

According to a further method of the present disclosure, the polymeric support material is tapered. The polymeric support material is tapered from the proximal end towards the distal end, in which the thickness of the polymeric support material is greater at the proximal end.

According to a configuration of the above method, the polymeric support material completely surrounds the cannula.

According to a configuration of the above method, the hub is a Luer-pressure fitting hub.

According to a further method of the present disclosure, a cannula assembly is formed. A cannula is provided having a proximal end and a distal end, polymeric support material, and a hub. Polymeric support material is located to substantially surround a portion of the cannula at or near the proximal end. The polymeric support material is located between the cannula and the hub. The hub and the polymeric support material and shrink wrapped to the cannula to securely attach the cannula, the polymeric support material, and the hub.

According to yet another method, a viral vector is delivered to a central nervous system of a subject. A cannula assembly and a syringe combination is provided and includes the cannula assembly and the syringe. The cannula assembly includes a cannula, polymeric support material and a hub. The cannula has a proximal end and a distal end. The polymeric support material substantially surrounds a portion of the cannula at or near the proximal end. The syringe includes a needle. The hub attaches the cannula assembly and the syringe. The polymeric support material is located between the cannula and the hub. The cannula, the polymeric support material and the hub are adhesively attached. The viral vector is provided. The viral vector is delivered to the central nervous system via the cannula assembly and the syringe combination.

In a further aspect of the above method, the viral vector is a recombinant viral vector.

In a further aspect of the above method, the viral vector is in a dosage of from about 0.5E9 to about 1.5E9 vg/mil. The viral vector may be in a dosage of from about 0.7E9 to about 1.3E9 vg/mil, or from about 0.7E9 to about 1.1E9 vg/mil.

In another aspect of the above method, the central nervous system is brain tissue or spinal cord.

In a further aspect of the above method, the subject has a neurological disorder. The neurological disorder may be meningitis, encephalitis, multiple sclerosis (MS), stroke, brain tumors, epilepsy, Alzheimer's disease, AIDS-related dementia, Parkinson's disease or Huntington's disease. More specifically, the neurological disorder may be Alzheimer's disease, Parkinson's disease or Huntington's disease.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of exemplary embodiments together with reference to the accompanying drawings. These drawings depict only exemplary embodiments, and are therefore not to be considered as limitations on the scope of the various embodiments or claims.

FIG. 1A is a cross-sectional side view of a cannula assembly according to one embodiment.

FIG. 1B is a front view of the cannula assembly of FIG. 1A.

FIG. 1C is an enlarged side view of area 1C taken from FIG. 1A with adhesive added.

FIG. 2A is a cross-sectional side view of a cannula assembly according to another embodiment.

FIG. 2B is an enlarged side view of area 2B taken from FIG. 2A.

FIG. 3 is a cross-sectional side view of a cannula assembly according to a further embodiment.

FIG. 4 is a perspective view of a syringe according to one embodiment.

FIG. 5A is a cross-sectional side view of a combination of the cannula assembly of FIG. 1 and the syringe of FIG. 4.

FIG. 5B is an enlarged side view of area 5B taken from FIG. 5A.

While the invention is susceptible to various modifications and alternative forms, specific implementations have been shown by way of example in the drawings and will be described in further detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

Various embodiments are described with reference to the attached figures, where like reference numerals are used throughout the figures to designate similar or equivalent elements. The figures are not drawn to scale and are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The various embodiments are not limited by the illustrated ordering of acts or events, as some acts may occur in different orders and/or concurrently with other acts or events. Furthermore, not all illustrated acts or events are required to implement a methodology in accordance with the present invention.

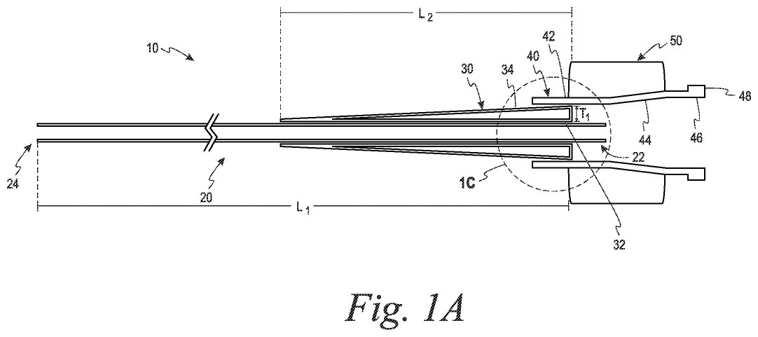

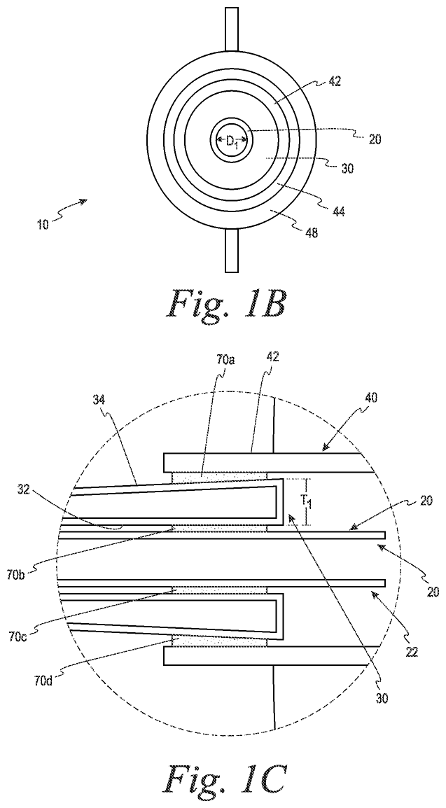

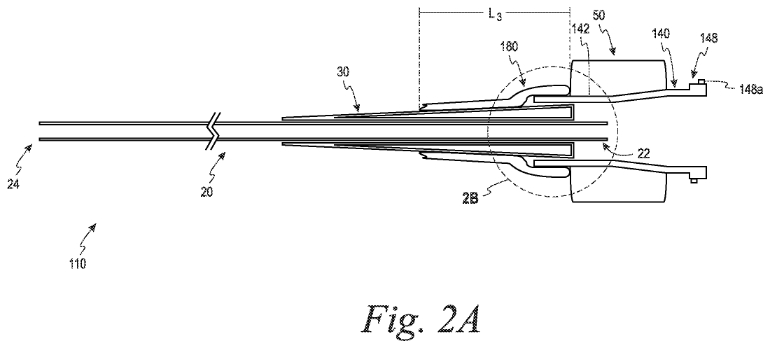

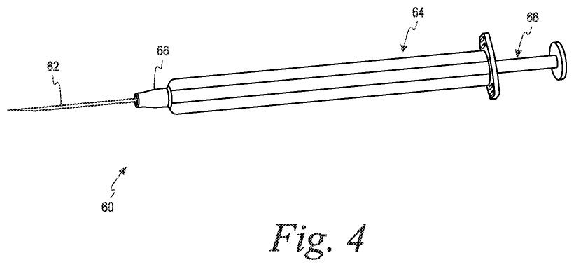

Referring initially to FIGS. 1A and 1B, a cannula assembly 10 is shown in a cross-sectional side view and a front view, respectively, according to one embodiment. The cross-hatching has been removed to enhance the clarity of FIG. 1A. The cannula assemblies of the present invention are configured to engage with a syringe (e.g., syringe 60 shown in FIG. 4) in one embodiment. The cannula assemblies assist in delivering liquid products to areas of the body including to brain tissue. The cannula assemblies enable more practical and leak-resistant connection to a delivery syringe for brain infusions. The cannula assemblies also assist in removing liquid or gathering samples from areas of the body including from brain tissue. It is contemplated that the cannula assemblies may be used in other aspects in other implementations.

Referring back to FIGS. 1A and 1B, the cannula assembly 10 includes a cannula 20, polymeric support material 30 and a hub 40. FIG. 1C is an enlarged side view of area 1C taken from FIG. 1A with adhesive added. The cannula 20 is a tube in one embodiment and is configured to be inserted into a body. The cannula 20 in one embodiment is a glass fiber cannula. It is contemplated that the cannula may be made of other materials such as metals. Non-limiting examples of metals that can be used in forming the cannula include stainless steel and titanium. It is also contemplated that polymeric materials with a desired stiffness may be used in forming the cannula.

The full length of the cannula 20 has been truncated in FIG. 1A for clarity and, thus, is not shown to scale. The cannula has a proximal end 22 and a distal end 24. The distal end 24 is configured to be inserted into a body. The cannula 20 can vary in length, but typically has a length L1 of from about 0.3 to about 1.5 meters. In one desired embodiment, the cannula 20 has a length L1 of from about 0.75 to about 1.25 meters. In another desired embodiment, the cannula 20 has a length L1 of from about 0.9 to about 1.1 meters.

The cannula 20 can vary in diameter, but typically has a diameter D1 (see FIG. 1B) of from about 0.25 to about 0.5 mm. In one desired embodiment, the cannula 20 has a diameter D1 of from about 0.3 to about 0.45 mm. In another desired embodiment, the cannula 20 has a diameter D1 of from about 0.3 to about 0.4 mm.

As shown in FIGS. 1A-1C, the polymeric support material 30 is located between the cannula 20 and the hub 40. Specifically, the polymer support material as best shown in FIG. 1C has an interior surface 32 located adjacent to the cannula 20 and an exterior surface 34 that is located adjacent to the hub 40. Referring to FIG. 1A, the polymeric support material 30 is located at or near the proximal end 22 of the cannula 20. As shown in FIGS. 1A-1C, the polymeric support material 30 substantially surrounds a portion of the cannula 20 at or near the proximal end 22. For example, the polymeric support material surrounds 70% or 85% of a portion of the cannula at or near the proximal end. In another embodiment, the polymeric support material surrounds 90% or 95% of a portion of the cannula at or near the proximal end. It is desirable for the polymeric support material 30 of FIGS. 1A-1C to completely surround a portion of the cannula 20 at or near the proximal end 22 as best shown in FIG. 1B.

The polymeric support material 30 assists in preventing or inhibiting stress risers. A stress riser is an abrupt change in flexibility likely to increase fractures of the cannula when the cannula is made of glass fibers. The polymeric support material 30 assists in changing the stress point by the use of distance, which assists in preventing or inhibiting breakage of the cannula 20.

The polymeric support material 30 can vary in length, but typically has a length L2 (see FIG. 1A) of from about 5 to about 40 cm in length. In one desired embodiment, the polymeric support material 30 has a length L2 of from about 10 to about 30 cm. In another desired embodiment, the polymeric support material 30 has a length L2 of from about 10 to about 20 cm, or from about 15 to about 20 cm.

The polymeric support material 30 is tapered in one embodiment from the proximal end 22 of the cannula 20 towards the distal end 24. Thickness T1 of the polymeric support material 30 is greater at or near the proximal end 22 of the cannula 20. The thickness T1 of the polymeric support material 30 is from about 1 mm to about 5 mm and, more specifically, from about 1.5 mm to about 4 mm, or from about 2 mm to about 4 mm.

In one embodiment, the polymeric support material 30 includes polytetrafluoroethylene (PTFE). The polymeric support material may be made of other polymeric materials or combinations of polymeric materials. Some other non-limiting materials that may be used in forming the polymeric support material include polyamides, fluoropolymers, polyolefins, PVC (polyvinyl chlorides), polyimides, PEEK (polyetheretherketones), or combinations thereof. It is also desirable for the polymeric support material to be generally clear or translucent so as to allow transmission of ultraviolet (UV) light if an UV adhesive is used. It is also desirable to have the material forming the polymeric support material to be compatible with adeno-associated viruses (AAV) (i.e., AAV viruses don't adhere to the material).

The hub 40 as shown in FIGS. 1A-1C is located adjacent to the exterior surface 34 of the polymeric support material 30. The hub 40 is configured to assist in attaching and securing the cannula 20 and a syringe (e.g., the syringe 60 in FIG. 4). The hub 40 also desirably provides a tight seal with a syringe, as will be discussed below, that assists in preventing or inhibiting leakage of any liquid material contained within the syringe or within the proximal end 22 of the cannula 20. The hub 40 also is desirably configured to reduce or effectively eliminate much of the dead space where any liquid could be unaccounted for or bubbles to accumulate.

The hub 40 as shown in FIG. 1A depicts a first generally horizontal section 42, a slightly upwardly tapered section 44, and a second generally horizontal section 46. The second generally horizontal section 46 includes an outer rim 48. The outer rim 48 strengthens the hub 40.

In one embodiment, the hub 40 is a Luer-pressure fitting hub. Some advantages of using a Luer-pressure fitting hub include ease and security of attachment. It is contemplated that other hubs may be used in the cannula assemblies.

The hub 40 may be made of materials including, but not limited to, polymeric materials. Some non-limiting examples of polymeric materials include, but are not limited to, polyolefins (e.g., polypropylenes). It is also desirable for the hub to be generally clear or translucent so as to allow transmission of UV light if an UV adhesive is used. It is also desired to have the material forming the hub to be compatible with adeno-associated viruses (AAV).

The cannula 20, the polymeric support material 30 and the hub 40 are adhesively attached in one embodiment. The adhesive may be applied and located within different areas to securely attach the cannula 20, the polymeric support material 30 and the hub 40. The adhesive is applied on at least one of the cannula, the polymeric support material, and the hub. It is contemplated that the adhesive may be applied on two or more of the cannula, the polymeric support material, and the hub. Some representative areas are shown in FIG. 1C with adhesive 70a-70d. The adhesive areas 70a, 70d are located between the hub 40 and the polymeric support material 30. The adhesive areas 70b, 70c are located between the polymeric support material 30 and the cannula 20. The adhesive permanently and securely attaches the cannula 20, the polymeric support material 30 and the hub 40.

In one embodiment, the adhesive is an UV-sensitive adhesive. In this embodiment, the adhesive is typically a liquid adhesive that is cured using UV light. The surface tension of the material that the liquid adhesive contacts assists in maintaining the positioning of the adhesive before curing. This may be performed in a single step in one method. In another method, the curing of the adhesive may be formed in a multi-step process.

A UV adhesive is an adhesive that typically works on an epoxy resin or an acrylic base. One UV adhesive is a radically-initiated UV adhesive based on an acrylate mixture (e.g., urethane, cyanoacrylate or silicone). Thus, UV adhesives include, but are not limited to, urethane acrylate adhesive compositions, cyanoacrylate adhesive compositions, and silicone acrylate adhesive compositions. UV adhesives based on acrylates are typically solvent-free and include one component. Another UV adhesive is a cationically-initiated UV adhesive based on epoxy resins. UV adhesives are marked by companies such as Bondic, RapidFix and Dymax.

To cure, a UV-sensitive adhesive requires a light source. This can come from pure sunlight, but also from UV LED lights and UV gas discharge lamps. However, LED light sources must be matched to the respective adhesive and are therefore available in different wavelengths. A UV-sensitive adhesive usually cures very quickly. For example, the curing of a UV-sensitive adhesive can occur between about 1 and about 10 seconds, and more specifically, from about 1 to about 5 seconds, and from about 1 to about 3 seconds. Typically, the more intense the light source, the faster the curing process. UV-sensitive adhesives may be adhesives that cure only when the user exposes it to UV light of a precisely defined wavelength.

The cannula 20, the polymeric support material 30 and the hub 40 are securely attached such that an individual cannot easily separate the components from each other. Thus, for example, the cannula 20, the polymeric support material 30 and the hub 40 are not attached by a press-fit. Thus, the cannula 20, the polymeric support material 30 and the hub 40 are formed in the absence of a press-fit.

In one embodiment, the cannula assembly is a non-detachable, closed sterile system. In one method, the product to be delivered using the cannula assembly does not contact an adhesive that is used to securely attach the cannula 20, the polymeric support material 30 and the hub 40. Similarly, in another method, fluid or other product to be removed the product to be delivered using the cannula assembly does not contact an adhesive that is used to securely attach the cannula 20, the polymeric support material 30 and the hub 40.

The cannula assembly 10 may further include a winged connection 50 adapted to tighten the connection between a syringe (e.g., syringe 60 in FIG. 4) and the remainder of the cannula assembly 10. In one process, the winged connection 50 may be tightened by the use of a thumb and a forefinger. To assist in grasping the winged connection 50, it may include a corrugated, concave area to generally correspond with a shape of a thumb or a finger.

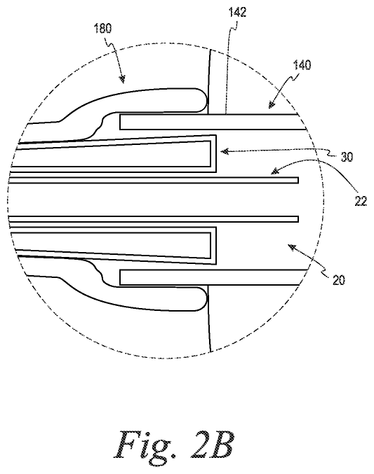

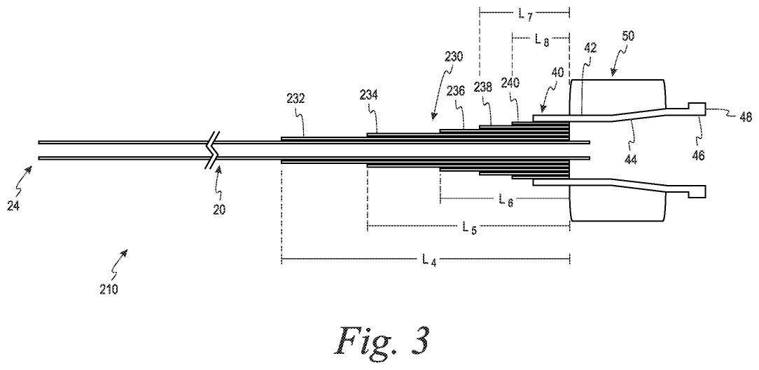

Referring to FIGS. 2A, 2B, a cannula assembly 110 is shown in a cross-sectional side view according to another embodiment. The cross-hatching has been removed in FIGS. 2A, 2B to enhance the clarity. The cannula assembly 110 includes the cannula 20, the polymeric support material 30, a hub 140, overlying tubing 180 and the winged connection 50.

The hub 140 is the same as the hub 40 described above except for outer rim 148. The outer rim 148 strengthens the hub 140 in a similar manner as the outer rim 48 to the hub 40. The outer rim 148 also includes an external thread formation 148a. The external thread formation 148a may be a single thread or a plurality of threads. The external thread formation 148a is configured to be securely attached with a component of a syringe having an internal thread formation. For example, the external thread formation 148a may be attached to a Hamilton connector ring of a syringe. Thus, the external thread formation 148a assists in securely attaching the cannula assembly 110 with a syringe (e.g., syringe 60 of FIG. 4).

The overlying tubing 180 is located on an exterior surface 142 of the hub 140 as shown in FIGS. 2A, 2B. The overlying tubing 180 in one embodiment is made of a flexible material. Non-limiting examples of materials that may be used in forming the overlying tubing 180 include, but are not limited to, polytetrafluoroethylene (PTFE). Some other non-limiting materials that may be used in forming the overlying tubing include polyamides, fluoropolymers, polyolefins, PVC (polyvinyl chlorides), polyimides, PEEK (polyetheretherketones), or combinations thereof. It is also desirable for the polymeric support material to be generally clear or translucent so as to allow transmission of UV light if an UV adhesive is used. The overlying tubing 180 is located generally above the proximal end 22 of the cannula 20. The overlying tubing 180 assists in absorbing and spreading the shear load to prevent or inhibit breakage of the cannula 20.

The overlying tubing 180 can vary in length, but typically has a length L3 (see FIG. 2A) of from about 5 to about 25 cm. In one desired embodiment, the overlying tubing 180 has a length L3 of from about 10 to about 20 cm. In another desired embodiment, the overlying support 180 has a length L3 of from about 10 to about 15 cm.

Referring to FIG. 3, a cannula assembly 210 is shown in a cross-sectional side view according to a further embodiment. The cross-hatching has been removed in FIG. 3 to enhance the clarity. The cannula assembly 210 includes a cannula 20, polymeric support material 230, the hub 40 and the winged connection 50. The polymeric support material 230 assists in preventing or inhibiting stress risers.

The polymeric support material 230 includes a plurality of polymeric supporting tube segments 232, 234, 236, 238, 240 that are successively stacked on top of each other. The lengths L4-L8 (see FIG. 3) of each of the plurality of polymeric supporting tube segments 232, 234, 236, 238, 240 are different. The length L4 of the polymeric supporting tube segment 232 is the longest, while the length L8 is the shortest. Each of the lengths of the polymeric supporting tube segments 232, 234, 236, 238, 240 gets progressively shorter. The length L4 of the polymeric supporting tube segment 232 is similar to the length L1 of the polymeric support material 30. It is contemplated that the number of polymeric supporting tube segments may be greater or lesser in number than depicted in FIG. 3.

As discussed above, the cannula assemblies are adapted to work in conjunction with a syringe for delivering or receiving fluids. One type of syringe that may be used in conjunction with the cannula assemblies is shown in FIG. 4 with the syringe 60. The syringe 60 includes a needle 62, a barrel assembly 64, a plunger assembly 66 and a male termination 68. The needle 62 is typically covered by a nut (not shown) to protect inadvertent contact with the needle 62. The syringe 60 may be referred to as a Hamilton syringe. Referring to FIG. 4, the syringe 60 is a Hamilton syringe. It is contemplated that other types of syringes may be used with the cannula assemblies of the present invention such as, for example, those manufactured by Setonic, Trident, Becton Dickenson, and Alibaba.

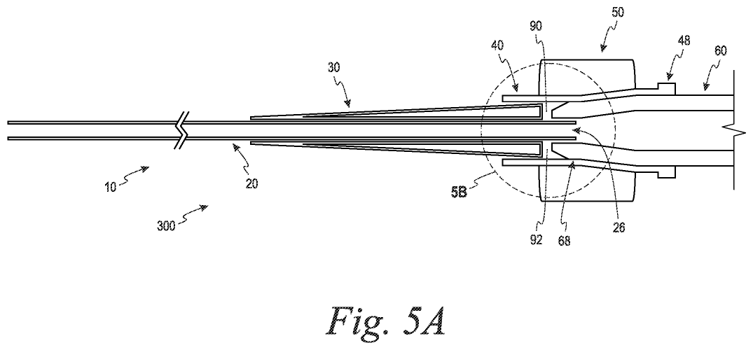

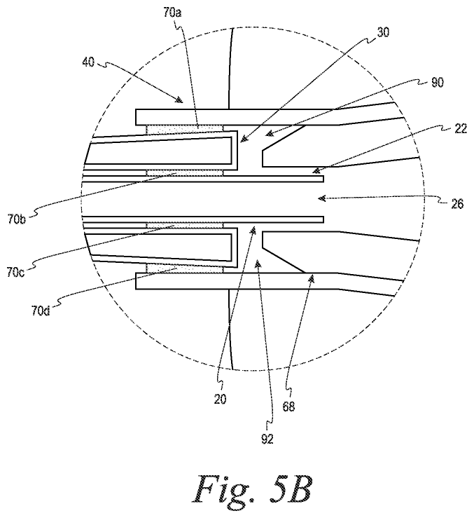

Referring to FIGS. 5A, 5B, a cannula assembly and syringe combination 300 is shown in a cross-sectional view. The cross-hatching has been removed to enhance the clarity in FIGS. 5A, 5B. The combination 300 includes the cannula assembly 10 and the syringe 60 described above. The needle 62 of the syringe is not shown for improved clarity. To reduce the dead space, the cannula 20 is extended sufficiently into the hub 40 to receive the syringe 60. The cannula 20 should extend a sufficient distance past the polymeric support material 30 such that process tolerances will not allow any potential adhesive to be applied near an opening 26 of the proximal end 22 of the cannula 20. The adhesive delivery and the processing step with UV light need to be precise to avoid blocking the cannula or creating leaks.

Since the proximal end 22 of the cannula 20 is located inside the lumen of the male termination 68 of the syringe 60, there is minimal dead space. As shown in FIGS. 5A, 5B, dead space 90, 92 is shown between the male termination 68, the hub 40 and polymeric support material 30. The hub 40 also desirably provides a tight seal with the male termination 68 of the syringe 60 to assist in preventing or inhibiting leakage of any liquid material contained within the syringe or within the proximal end 22 of the cannula 20.

It is contemplated that a combination may be formed using the cannula assemblies 110, 210 with the syringe 60 or with other syringes.

According to yet another method, a viral vector is delivered to a central nervous system of a subject. A cannula assembly and a syringe combination is provided and includes the cannula assembly and the syringe. The cannula assembly includes a cannula, polymeric support material and a hub. The cannula has a proximal end and a distal end. The polymeric support material substantially surrounds a portion of the cannula at or near the proximal end. The syringe includes a needle. The hub attaches the cannula assembly and the syringe. The polymeric support material is located between the cannula and the hub. The cannula, the polymeric support material and the hub are adhesively attached. The viral vector is provided. The viral vector is delivered to the central nervous system via the cannula assembly and the syringe combination.

Non-limiting examples of agents and therapeutic devices that may be delivered through a cannula assembly and syringe combination (e.g., the cannula assembly and syringe combination 300) as described herein include, but are not limited to, drugs, nanoparticles, biological agents (e g., cells, virus, etc.).

In one embodiment, the vector can be, but is not limited to, a nonviral vector or a viral vector. In one embodiment of any aspect, the vector is a DNA or RNA virus. Non-limiting examples of a viral vector include an AAV vector, an adenovirus vector, a lentivirus vector, a retrovirus vector, a herpesvirus vector, an alphavirus vector, a poxvirus vector, a baculovirus vector, and a chimeric virus vector. Non-limiting examples of AAV include AAV1, AAV2, AAV3, AAV4, AAV5, AAV6, AAV7, AAV8, AAV9, AAV10, AAV 11, AAV12, and any chimeras thereof. In some embodiments, AAV are AAV rhesus monkey serotype (AAV rh). Non limiting examples of AAV rh serotypes include AAV rh8, AAV rh10, AAV rh20, AAV rh74, AAV rh39, AAV rh43, AAV rh38, AAV rh40, AAV rh2, AAV rh25, AAV rh57, AAV rh50, AAV rh49, AAV rh58, AAV rh61, AAV rh52, AAV rh53, AAV rh51, AAV rh64, AAV rh8, AAV rh1, AAV rh62, AAV rh48, AAV rh54, AAV rh55, AAV rh35, AAV rh37, AAV rh36, AAV rh13, AAV rh32, AAV rh33, and AAV rh34.

Any viral vector that is known in the art can be used with the cannula assembly and syringe combination. Examples of such viral vectors include, but are not limited to, vectors derived from: Adenoviridae; Birnaviridae; Bunyaviridae; Caliciviridae; Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group); Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picomaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

An effective amount of a viral vector (e.g., a recombinant viral vector (rAAV)) is an amount sufficient to target infect an animal, or target a desired tissue. In some embodiments, an effective amount of a viral vector (e.g., a recombinant viral vector (rAAV)) is an amount sufficient to produce a stable somatic transgenic animal model. The effective amount will depend primarily on factors such as the species, age, weight, health of the subject, and the tissue to be targeted, and may thus vary among animal and tissue.

In some embodiments, a dose of a viral vector (e.g., a recombinant viral vector (rAAV)) is administered to a subject no more than once per calendar day (e.g., a 24-hour period). In some embodiments, a dose of viral vector is administered to a subject no more than once per 2, 3, 4, 5, 6, or 7 calendar days. In some embodiments, a dose of viral vector is administered to a subject no more than once per calendar week (e.g., 7 calendar days). In some embodiments, a dose of viral vector is administered to a subject no more than bi-weekly (e.g., once in a two calendar week period). In some embodiments, a dose of viral vector is administered to a subject no more than once per calendar month (e.g., once in 30 calendar days). In some embodiments, a dose of viral vector is administered to a subject no more than once per six calendar months. In some embodiments, a dose of viral vector is administered to a subject no more than once per calendar year (e.g., 365 days or 366 days in a leap year).

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for neuronal degradation or functionality among others. The dosage can be determined by a physician and adjusted, as necessary, to suit observed effects of the treatment.

In a further aspect of the above method, the viral vector is in a dosage of from about 0.5E9 to about 1.5E9 vg/μl (from about 0.5E12 to about 1.5E12 vg/mil). The viral vector may be in a dosage of from about 0.7E9 to about 1.3E9 vg/μl (from about 0.7E12 to about 1.3E12 vg/mil). In another embodiment, the viral vector may be in a dosage of from about 0.7E9 to about 1.1E9 vg/μl (from about 0.7E12 to about 1.1E12 vg/mil). The viral vector may be delivered to a subject. The desired dosage should be in range between a minimally effective dose at the low end to a less than a mildly toxic level.

The dose calculation can be complicated, but is mandated by the tissue reaction to products delivered directly into the parenchyma of the striatum or another part of the brain. If the concentration is too weak (and the volume too small) there is no therapeutic effect. If more concentrated, the product may be effective. If the concentration is too high, it invokes an inflammatory reaction and if sufficiently strong, it kills the cells at the injection site. After the dosage concentration is determined, the volume of that dilution is calculated that will be injected into each of the 4 striatal lobes. In humans and non-human primates (NHPs), the volumes are individually measured using MRI (2× caudates and 2× putamens). The volume of product to be injected into each lobe is calculated as a percentage of the measured organ volume. For example, this percentage can range from about 15% to about 50%. A higher percentage may be used (e.g., 35 to 50%) if there is inadequate perfusion into the target lobes. Typical healthy volumes for the putamen and caudate in human subjects are about 3.57 and about 2.73 cm³ respectively, and about 0.55 and about 0.41 cm³ in NHPs.

In mice, a standard volume for the striatum may be used. In mice, the striatum measures about 20 to about 37 mm³. For example, the volume delivered to a mouse may be 2 μL or 4 μL. In another example, the volume delivered to NHPs would be in a volume range of from about 20 μL to about 250 μL. In a further example, the volume delivered to humans would be in a volume range of from about 140 μL to about 1 mL.

The dosages are delivered using the cannula assemblies of the present invention. Deliveries by a needle, for example, into the parenchyma of the brain requires overcoming the static fluid pressure of the tissue. Thus, in one method, very slow rates of injection are used initially with two stepped up rates to complete the delivery. This is generally referred to as “convection enhanced delivery.” It is contemplated that other modified versions of delivering, including other three step infusion rates, may be used.

Viral vectors are delivered to a subject using the cannula assembly and syringe combination. For example, recombinant viral vector preferably suspended in a physiologically compatible carrier (i.e., in a composition), may be administered to a subject, i.e., host animal, such as a human, mouse, rat, cat, dog, sheep, rabbit, horse, cow, goat, pig, guinea pig, hamster, chicken, turkey, or a non-human primate (e.g., Macaque). In some embodiments, a host animal does not include a human.

Subjects to which the methods of the instant disclosure are applicable include veterinary subjects (e.g., dogs, cats, horses, etc.) and research animal subjects (e.g., mice, rats, rabbits, pigs, goats, sheep, primates, etc.), as well as human subjects. The methods are applicable to all primates, including simians. In some embodiments, the methods are applied to humans. In other embodiments, the methods are applied to non-human primates.

Any desired area of a subject may be targeted according to the methods described herein. In some instances, the desired area may be tissue including, but not limited to, a tissue of endodermal origin, a tissue of ectodermal origin, and a tissue mesodermal origin. Neural tissues are typically targeted. In some instances, neural tissues of the central nervous system (CNS) may be targeted including, for example, tissues of the brain and tissues of the spinal cord. In some instances, neural tissues of the peripheral nervous system may be targeted. It is contemplated that other tissue may be targeted according to the methods described herein.

In some instances, the methods may be applied for effective delivery/localization of an agent to a region of interest in the mammalian nervous system, including the central nervous system or the peripheral nervous system. Essentially any region of interest of the nervous system may be targeted according to the methods as described herein including, but not limited to, the brain, the spinal cord, the spinal ganglia, etc.

In some instances, the methods may be applied for effective delivery/localization of an agent to a region of interest in the mammalian brain. Essentially any region of interest of the brain may be targeted according to the methods as described herein.

Viral vectors described herein can be directly injected, into any region of the brain, such as, for example, occipital lobe, temporal lobe, parietal lobe, frontal lobe, cerebral cortex, cerebellum, hypothalamus, thalamus, pituitary gland, pineal gland, amygdala, hippocampus and the mid-brain.

In some instances, one or more brain lobes or a particular area within a brain lobe may be targeted including, but not limited to, the frontal lobe (either the entire frontal lobe or portions thereof including, but not limited to, Superior Frontal, Rostral Middle Frontal, Caudal Middle Frontal, Pars Opercularis, Pars Triangularis, and Pars Orbitalis, Lateral Orbitofrontal, Medial Orbitofrontal, Precentral, Paracentral, Frontal Pole, combinations thereof, and the like), parietal lobe (either the entire parietal lobe or portions thereof including, but not limited to, Superior Parietal, Inferior Parietal, Supramarginal, Postcentral, Precuneus, combinations thereof, and the like), temporal lobe (either the entire temporal lobe or portions thereof including, but not limited to, Superior Temporal, Middle Temporal, Infenor Temporal, Banks of the Superior Temporal Sulcus, Fusiform, Transverse Temporal, Entorhinal, Temporal Pole, Parahippocampal, combinations thereof, and the like) and occipital lobe (either the entire occipital lobe or portions thereof including, but not limited to. Lateral Occipital, Lingual, Cuneus, Pericalcarine, combinations thereof, and the like).

In some instances, one or more brain structures or a particular area within a brain structure may be targeted including, but not limited to, Hindbrain structures (e.g., Myelencephalon structures (e.g., Medulla oblongata, Medullary pyramids, Olivary body, Inferior olivary nucleus, Respiratory center, Cuneate nucleus, Gracile nucleus, Intercalated nucleus, Medullary cranial nerve nuclei, Inferior salivatory nucleus, Nucleus ambiguous, Dorsal nucleus of vagus nerve, Hypoglossal nucleus, Solitary nucleus, etc.), Metencephalon structures (e.g., Pons, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centres, Pneumotaxic centre, Apneustic centre, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Fourth ventricle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, etc.)), Midbrain structures (e.g., Tectum, Corpora quadrigemina, inferior colliculi, superior colliculi, Pretectum, Tegmentum, Periaqueductal gray, Parabrachial area, Medial parabrachial nucleus, Lateral parabrachial nucleus, Subparabrachial nucleus (Kolliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (Ill), Trochlear nucleus (IV), Mesencephalic duct (cerebral aqueduct, aqueduct of Sylvius), etc.), Forebrain structures (e.g., Diencephalon, Epithalamus structures (e.g., Pineal body, Habenular nuclei, Stria medullares, Taenia thalami, etc.) Third ventricle, Thalamus structures (e.g., Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, etc.), Hypothalamus structures (e.g., Anterior, Medial area, Parts of preoptic area, Medial preoptic nucleus, Suprachiasmatic nucleus, Paraventricular nucleus, Supraoptic nucleus (mainly), Anterior hypothalamic nucleus, Lateral area, Parts of preoptic area, Lateral preoptic nucleus, Anterior part of Lateral nucleus, Part of supraoptic nucleus, Other nuclei of preoptic area, median preoptic nucleus, periventricular preoptic nucleus, Tuberal, Medial area, Dorsomedial hypothalamic nucleus, Ventromedial nucleus, Arcuate nucleus, Lateral area, Tuberal part of Lateral nucleus, Lateral tuberal nuclei, Posterior, Medial area, Mammillary nuclei (part of mammillary bodies), Posterior nucleus, Lateral area, Posterior part of Lateral nucleus, Optic chiasm, Subfomical organ, Periventricular nucleus, Pituitary stalk, Tuber cinereum, Tuberal nucleus, Tuberomammillary nucleus, Tuberal region, Mammillary bodies, Mammillary nucleus, etc.), Subthalamus structures (e.g., Thalamic nucleus, Zona incerta, etc.), Pituitary gland structures (e.g., neurohypophysis, Pars intermedia (Intermediate Lobe), adenohypophysis, etc.), Telencephalon structures, white matter structures (e.g., Corona radiata, Internal capsule, External capsule, Extreme capsule, Arcuate fasciculus, Uncinate fasciculus, Perforant Path, etc.), Subcortical structures (e.g., Hippocampus (Medial Temporal Lobe), Dentate gyrus, Comu ammonis (CA fields), Comu ammonis area 1, Comu ammonis area 2, Comu ammonis area 3, Comu ammonis area 4, Amygdala (limbic system) (limbic lobe), Central nucleus (autonomic nervous system), Medial nucleus (accessory olfactory system), Cortical and basomedial nuclei (main olfactory system), Lateral[disambiguation needed] and basolateral nuclei (frontotemporal cortical system), Claustrum, Basal ganglia, Striatum, Dorsal striatum (aka neostriatum), Putamen, Caudate nucleus, Ventral striatum, Nucleus accumbens, Olfactory tubercle, Globus pallidus (forms nucleus lentiformis with putamen), Subthalamic nucleus, Basal forebrain, Anterior perforated substance, Substantia innominata, Nucleus basalis, Diagonal band of Broca, Medial septal nuclei, etc.), Rhinencephalon structures (e.g., Olfactory bulb, Piriform cortex, Anterior olfactory nucleus, Olfactory tract, Anterior commissure, Uncus, etc.), Cerebral cortex structures (e.g., Frontal lobe, Cortex, Primary motor cortex (Precentral gyrus, Ml), Supplementary motor cortex, Premotor cortex, Prefrontal cortex, Gyri, Superior frontal gyrus, Middle frontal gyrus, Inferior frontal gyrus, Brodmann areas: 4, 6, 8, 9, 10, 11, 12, 24, 25, 32, 33, 44, 45, 46, 47, Parietal lobe, Cortex, Primary somatosensory cortex (S1), Secondary somatosensory cortex (S2), Posterior parietal cortex, Gyri, Postcentral gyrus (Primary somesthetic area), Other, Precuneus, Brodmann areas 1, 2, 3 (Primary somesthetic area); 5, 7, 23, 26, 29, 31, 39, 40, Occipital lobe, Cortex, Primary visual cortex (V1), V2, V3, V4, V5/MT, Gyri, Lateral occipital gyrus, Cuneus, Brodmann areas 17 (V1, primary visual cortex); 18, 19, Temporal lobe, Cortex, Primary auditory cortex (A1), secondary auditory cortex (A2), Inferior temporal cortex, Posterior inferior temporal cortex, Superior temporal gyrus, Middle temporal gyrus, Inferior temporal gyrus, Entorhinal Cortex, Perirhinal Cortex, Parahippocampal gyrus, Fusiform gyrus, Brodmann areas: 9, 20, 21, 22, 27, 34, 35, 36, 37, 38, 41, 42, Medial superior temporal area (MST), Insular cortex, Cingulate cortex, Anterior cingulate, Posterior cingulate, Retrosplenial cortex, Indusium griseum, Subgenual area 25, and Brodmann areas 23, 24; 26, 29, 30 (retrosplenial areas); 31, 32, etc.)).

In some instances, one or more neural pathways or a particular portion of a neural pathway may be targeted including, but not limited to, neural pathways of those brain lobes and structures described above, Superior Longitudinal Fasciculus. Arcuate fasciculus, Cerebral peduncle, Corpus callosum. Pyramidal or corticospinal tract, Major dopamine pathways dopamine system, Mesocortical pathway. Mesolimbic pathway, Nigrostriatal pathway, Tuberoinfundibular pathway, Serotonin Pathways serotonin system. Raphe Nuclei, Norepinephrine Pathways, and Locus coeruleus, etc.

Diseased neural tissues that may be targeted include, but are not limited to, neural tissue disease due to one or more of meningitis, encephalitis, multiple sclerosis (MS), stroke, brain tumors, epilepsy. Alzheimer's disease, AIDS-related dementia, Parkinson's disease and Huntington's disease.

Delivery of the compositions to a mammalian subject may be by, for example, any known means of delivering to a desire site, e.g., the central nervous system (CNS). It may be desirable to deliver the composition to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Any composition described herein may be delivered directly to the CNS or brain by injection into, for example, the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection. In some embodiments, compositions as described in the disclosure are administered by intravenous injection. In some embodiments, compositions as described in the disclosure are administered by intraspinal injection. In some embodiments, compositions as described in the disclosure are administered by intracerebro ventricular injection. In some embodiments, compositions are administered by intracerebral injection. In some embodiments, compositions are administered by intrathecal injection. In some embodiments, compositions are administered by intrastriatal injection. In some embodiments, compositions are delivered by intracranial injection. In some embodiments, compositions are delivered by cistema magna injection. In some embodiments, compositions are delivered by cerebral lateral ventricle injection.

The CNS includes, but is not limited to, certain regions of the CNS, neural pathways, somatosensory systems, visual systems, auditory systems, nerves, neuro endocrine systems, neuro vascular systems, brain neurotransmitter systems, and dural meningeal system.

Exemplary regions of the CNS include, but are not limited to, Myelencephalon; Medulla oblongata; Medullary pyramids; Olivary body; Inferior olivary nucleus; Rostral ventrolateral medulla; Caudal ventrolateral medulla; Solitary nucleus (Nucleus of the solitary tract); Respiratory center-Respiratory groups Dorsal respiratory group; Ventral respiratory group or Apneustic centre Pre-Bötzinger complex; Botzinger complex; Retrotrapezoid nucleus; Nucleus retrofacialis; Nucleus retroambiguus; Nucleus para-ambiguus; Paramedian reticular nucleus; Gigantocellular reticular nucleus; Parafacial zone; Cuneate nucleus; Gracile nucleus; Perihypoglossal nuclei; Intercalated nucleus; Prepositus nucleus; Sublingual nucleus; Area postrema; Medullary cranial nerve nuclei; Inferior salivatory nucleus; Nucleus ambiguus; Dorsal nucleus of vagus nerve; Hypoglossal nucleus; Chemoreceptor trigger zone; Metencephalon; Pons; Pontine nuclei; Pontine cranial nerve nuclei; Chief or pontine nucleus of the trigeminal nerve sensory nucleus; Motor nucleus for the trigeminal nerve; Abducens nucleus (VI); Facial nerve nucleus (VII); Vestibulocochlear nuclei (vestibular nuclei and cochlear nuclei) (VIII); Superior salivatory nucleus; Pontine tegmentum; Pontine micturition center (Barrington's nucleus); Locus coeruleus; Pedunculopontine nucleus; Laterodorsal tegmental nucleus; Tegmental pontine reticular nucleus; Nucleus incertus; Parabrachial area; Medial parabrachial nucleus; Lateral parabrachial nucleus; Subparabrachial nucleus (Kölliker-Fuse nucleus); Pontine respiratory group; Superior olivary complex; Medial superior olive; Lateral superior olive; Medial nucleus of the trapezoid body; Paramedian pontine reticular formation; Parvocellular reticular nucleus; Caudal pontine reticular nucleus; Cerebellar peduncles; Superior cerebellar peduncle; Middle cerebellar peduncle; Inferior cerebellar peduncle; Fourth ventricle; Cerebellum Cerebellar vermis; Cerebellar hemispheres; Anterior lobe; Posterior lobe; Flocculonodular lobe; Cerebellar nuclei; Fastigial nucleus; Interposed nucleus; Globose nucleus; Emboliform nucleus; Dentate nucleus; Midbrain (mesencephalon); Tectum Corpora quadrigemina; Inferior colliculi; Superior colliculi; Pretectum; Tegmentum Periaqueductal gray; Rostral interstitial nucleus of medial longitudinal fasciculus; Midbrain reticular formation; Dorsal raphe nucleus; Red nucleus; Ventral tegmental area; Parabrachial pigmented nucleus; Paranigral nucleus; Rostromedial tegmental nucleus; Caudal linear nucleus; Rostral linear nucleus of the raphe; Interfascicular nucleus; Substantia nigra; Pars compacta; Pars reticulata; Interpeduncular nucleus; Cerebral peduncle; Crus cerebri; Mesencephalic cranial nerve nuclei; Oculomotor nucleus (III); Edinger-Westphal nucleus; Trochlear nucleus (IV); Mesencephalic duct (cerebral aqueduct, aqueduct of Sylvius); Forebrain (prosencephalon); Diencephalon; Epithalamus; Pineal body (pineal gland); Habenular nuclei; Stria medullaris; Taenia thalami; Third ventricle; Subcommissural organ; Thalamus; Anterior nuclear group; Anteroventral nucleus (a.k.a. ventral anterior nucleus); Anterodorsal nucleus; Anteromedial nucleus; Medial nuclear group; Medial dorsal nucleus; Midline nuclear group; Paratenial nucleus; Reuniens nucleus; Rhomboidal nucleus; Intralaminar nuclear group; Centromedian nucleus; Parafascicular nucleus; Paracentral nucleus; Central lateral nucleus; Lateral nuclear group; Lateral dorsal nucleus; Lateral posterior nucleus; Pulvinar; Ventral nuclear group Ventral anterior nucleus; Ventral lateral nucleus; Ventral posterior nucleus; Ventral posterior lateral nucleus; Ventral posterior medial nucleus; Metathalamus; Medial geniculate body; Lateral geniculate body; Thalamic reticular nucleus; Hypothalamus (limbic system) (HPA axis); Anterior Medial area Parts of preoptic area; Medial preoptic nucleus INAH 1; INAH 2; INAH 3; INAH 4; Median preoptic nucleus; Suprachiasmatic nucleus; Paraventricular nucleus; Supraoptic nucleus (mainly); Anterior hypothalamic nucleus; Lateral area; Parts of preoptic area; Lateral preoptic nucleus; Anterior part of Lateral nucleus; Part of supraoptic nucleus; Other nuclei of preoptic area; Median preoptic nucleus; Periventricular preoptic nucleus; Tuberal Medial area; Dorsomedial hypothalamic nucleus; Ventromedial nucleus; Arcuate nucleus; Lateral area Tuberal part of Lateral nucleus; Lateral tuberal nuclei; Posterior Medial area Mammillary nuclei (part of mammillary bodies); Posterior nucleus; Lateral area Posterior part of Lateral nucleus; Surface Median eminence; Mammillary bodies; Pituitary stalk (infundibulum); Optic chiasm; Subfomical organ; Periventricular nucleus; Tuber cinereum; Tuberal nucleus; Tuberomammillary nucleus; Tuberal region; Mammillary nucleus; Subthalamus (HPA axis); Subthalamic nucleus; Zona incerta; Pituitary gland (HPA axis); Neurohypophysis; Pars intermedia (Intermediate Lobe); Adenohypophysis; Telencephalon (cerebrum); Cerebral hemispheres; White matter; Centrum semiovale; Corona radiata; Internal capsule; External capsule; Extreme capsule; Subcortical; Hippocampus (Medial Temporal Lobe); Dentate gyrus; Comu ammonis (CA fields); Comu ammonis area 1 (CA1); Comu ammonis area 2 (CA2); Comu ammonis area 3 (CA3); Comu ammonis area 4 (CA4); Amygdala (limbic system) (limbic lobe); Central nucleus (autonomic nervous system); Medial nucleus (accessory olfactory system); Cortical and basomedial nuclei (main olfactory system); Lateral and basolateral nuclei (frontotemporal cortical system); Extended amygdala; Stria terminalis Bed nucleus of the stria terminalis; Claustrum; Basal ganglia; Striatum Dorsal striatum (a.k.a. neostriatum); Putamen; Caudate nucleus; Ventral striatum; Nucleus accumbens; Olfactory tubercle; Globus pallidus (forms nucleus lentiformis with putamen); Ventral pallidum; Subthalamic nucleus; Basal forebrain; Anterior perforated substance; Substantia innominata; Nucleus basalis; Diagonal band of Broca; Septal nuclei; Medial septal nuclei; Lamina terminalis; Vascular organ of lamina terminalis; Rhinencephalon (paleocortex); Olfactory bulb; Olfactory tract; Anterior olfactory nucleus; Piriform cortex; Anterior commissure; Uncus; Periamygdaloid cortex; Cerebral cortex (neocortex); Frontal lobe; Cortex Primary motor cortex (Precentral gyrus, Ml); Supplementary motor cortex; Premotor cortex; Prefrontal cortex; Orbitofrontal cortex; Dorsolateral prefrontal cortex; Gyri Superior frontal gyrus; Middle frontal gyrus; Inferior frontal gyrus; Brodmann areas: 4, 6, 8, 9, 10, 11, 12, 24, 25, 32, 33, 44, 45, 46, 47; Parietal lobe Cortex Primary somatosensory cortex (S1); Secondary somatosensory cortex (S2); Posterior parietal cortex; Gyri Postcentral gyrus (Primary somesthetic area); Brodmann areas 1, 2, 3 (Primary somesthetic area); 5, 7, 23, 26, 29, 31, 39, 40; Occipital lobe Cortex Primary visual cortex (V1), V2, V3, V4, V5/MT; Gyri Lateral occipital gyrus; Brodmann areas 17 (V1, primary visual cortex); 18, 19; Temporal lobe Cortex Primary auditory cortex (A1); Secondary auditory cortex (A2); Inferior temporal cortex; Posterior inferior temporal cortex; Gyri Superior temporal gyrus; Middle temporal gyrus; Inferior temporal gyrus; Entorhinal cortex; Perirhinal cortex; Parahippocampal gyrus; Fusiform gyrus; Brodmann areas: 20, 21, 22, 27, 34, 35, 36, 37, 38, 41, 42; Insular cortex; Cingulate cortex Anterior cingulate; Posterior cingulate; Retrosplenial cortex; Indusium griseum; Subgenual area 25; and Brodmann areas 23, 24; 26, 29, 30 (retrosplenial areas); 31, and 32.

Exemplary neural pathways include, but are not limited to, Superior longitudinal fasciculus Arcuate fasciculus; Uncinate fasciculus; Perforant pathway; Thalamocortical radiations; Corpus callosum; Anterior commissure; Amygdalofugal pathway; Interthalamic adhesion; Posterior commissure; Habenular commissure; Fornix; Mammillotegmental; fasciculus; Incertohypothalamic pathway; Cerebral peduncle; Medial forebrain bundle; Medial longitudinal fasciculus; Myoclonic triangle; Solitary tract; Major dopaminergic pathways from dopaminergic cell groups; Mesocortical pathway; Mesolimbic pathway; Nigrostriatal pathway; Tuberoinfundibular pathway; Serotonergic pathways Raphe Nuclei; Norepinephrine Pathways Locus coeruleus and other noradrenergic cell groups; Epinephrine pathways from adrenergic cell groups; Glutamate and acetylcholine pathways from mesopontine nuclei; Motor systems/Descending fibers; Extrapyramidal system; Pyramidal tract; Corticospinal tract; or Cerebrospinal fibers; Lateral corticospinal tract; Anterior corticospinal tract; Corticopontine fibers; Frontopontine fibers; Temporopontine fibers; Corticobulbar tract; Corticomesencephalic tract; Tectospinal tract; Interstitiospinal tract; Rubrospinal tract; Rubro-olivary tract; Olivocerebellar tract; Olivospinal tract; Vestibulospinal tract; Lateral vestibulospinal tract; Medial vestibulospinal tract; Reticulospinal tract; Lateral raphespinal tract; Alpha system; and Gamma system.

Exemplary somatosensory systems include, but are not limited to, Dorsal column-medial lemniscus pathway Gracile fasciculus; Cuneate fasciculus; Medial lemniscus; Spinothalamic tract; Lateral spinothalamic tract; Anterior spinothalamic tract; Spinomesencephalic tract; Spinocerebellar tract; Spino-olivary tract; and Spinoreticular tract.

Exemplary visual systems include, but are not limited to, Optic tract; Optic radiation; Retinohypothalamic and tract.

Exemplary auditory systems include, but are not limited to, Medullary striae of fourth ventricle; Trapezoid body; and Lateral lemniscus.

Exemplary nerves include, but are not limited to, Brain stem Cranial nerves Terminal (0); Olfactory (I); Optic (II); Oculomotor (III); Trochlear (IV); Trigeminal (V); Abducens (VI); Facial (VII); Vestibulocochlear (VIII); Glossopharyngeal (IX); Vagus(X); Accessory (XI); and Hypoglossal (XII).

Exemplary neuro endocrine systems include, but are not limited to, Hypothalamic-pituitary hormones; HPA axis; HPG axis; HPT axis; and GHRH-GH.

Exemplary neuro vascular systems include, but are not limited to, Middle cerebral artery; Posterior cerebral artery; Anterior cerebral artery; Vertebral artery; Basilar artery; Circle of Willis (arterial system); Blood-brain barrier; Glymphatic system; Venous systems; and Circumventricular organs.

Exemplary brain neurotransmitter systems include, but are not limited to, Noradrenaline system; Dopamine system; Serotonin system; Cholinergic system; GABA; Neuropeptides Opioid peptides; Endorphins; Enkephalins; Dynorphins; Oxytocin; and Substance P.

Exemplary dural meningeal system include, but are not limited to, Brain-cerebrospinal fluid barrier; Meningeal coverings Dura mater; Arachnoid mater; Pia mater; Epidural space; Subdural space; Subarachnoid space Arachnoid septum; Superior cistern; Cistern of lamina terminalis; Chiasmatic cistern; Interpeduncular cistern; Pontine cistern; Cistema magna; Spinal subarachnoid space; Ventricular system; Cerebrospinal fluid; Third ventricle; Fourth ventricle; Lateral ventricles Angular bundle; Anterior horn; Body of lateral ventricle; Inferior horn; Posterior horn Calcar avis; and Subventricular zone.

The “PNS” refers to the nerves and ganglia outside the brain and spinal cord. The main function of the PNS is to connect the CNS to the limbs and organs, essentially serving as a relay between the brain and spinal cord and the rest of the body. Unlike the CNS, the PNS is not protected by the vertebral column and skull, or by the blood-brain barrier, which leaves it exposed to, e.g., toxins and mechanical injuries.

The peripheral nervous system (PNS) is divided into the somatic nervous system and the autonomic nervous system. In the somatic nervous system, the cranial nerves are part of the PNS with the exception of the optic nerve (cranial nerve II), along with the retina. The second cranial nerve is not a true peripheral nerve but a tract of the diencephalon. Cranial nerve ganglia originated in the CNS. However, the remaining ten cranial nerve axons extend beyond the brain and are therefore considered part of the PNS. The autonomic nervous system exerts involuntary control over smooth muscle and glands. The connection between CNS and organs allows the system to be in two different functional states: sympathetic and parasympathetic.

As used herein, “neurological disease or disorder” can refer to any disease, disorder, or condition affecting or associated with the nervous system, i.e., those that affect the central nervous system (brain and spinal cord), the peripheral nervous system (peripheral nerves and cranial nerves), and the autonomic nervous system (parts of which are located in both central and peripheral nervous systems). More than 600 neurological diseases have been identified in humans. By way of non-limiting examples, the neurological disease or disorder includes Alzheimer's disease, Parkinson's disease, Huntington's disease, Canavan disease, Leigh's disease, spinal cerebral ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, ophthalmic diseases and disorders, Tay-Sachs disease, Lesch-Nyan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, a psychosexual disorder, a sleeping disorder, a pain disorder, and/or a eating or weight disorder. In some embodiments, the neurological disease or disorder is a central nervous system (CNS) disease or disorder, e.g., Huntington's disease, Parkinson's disease, or Alzheimer's disease.

In one aspect, the invention provides methods for treating neurological disorders involving the cortex, referred to herein as “cortical neurological disorders.” The methods involve delivery of viral vectors described herein, or composition thereof to the CNS or PNS. Preferred cortical neurological disorders are those that involve large areas of the cortex, preferably more than one functional area of the cortex, preferably more than one lobe of the cortex, and up to and including the entire cortex. Preferred cortical neurological disorders include, but are not limited to, traumatic brain injury; stroke; enzymatic dysfunction disorders; psychiatric disorders, including post-traumatic stress syndrome; neurodegenerative diseases, including Huntington's disease, Parkinson's disease and Alzheimer's disease; epilepsy; and cognitive disorders, including dementias, autism, and depression. Preferred enzymatic dysfunction disorders include, but are not limited to, leukodystrophies, including Canavan's disease, and lysosomal storage diseases (LSD), including Niemann-Pick disease, Gaucher disease, Batten disease, Fabry disease and Pompe disease.

“Cortical neurological disorder”, as used herein, refers to a neurological disorder involving the cortex. Cortical neurological disorders are neurological disorders that: (i) involve a population of cells in the cortex that is directly anatomically connected to the thalamus, and/or (ii) involve a population of cells that is directly anatomically connected to the cortical cell population in (i).

Preferred cortical neurological disorders are those that involve large areas of the cortex, preferably more than one functional area of the cortex, preferably more than one lobe of the cortex, and up to and including the entire cortex. Preferred cortical neurological disorders include, but are not limited to, traumatic brain injury; stroke; enzymatic dysfunction disorders; psychiatric disorders, including post-traumatic stress syndrome; neurodegenerative diseases, including Huntington's disease, Parkinson's disease and Alzheimer's disease; epilepsy; and cognitive disorders, including dementias, autism, and depression. Preferred enzymatic dysfunction disorders include, but are not limited to leukodystrophies, including Canavan's disease, and lysosomal storage diseases (LSD), including Niemann-Pick disease, Gaucher disease, Batten disease, Fabry disease and Pompe disease. This list of disorders is exemplary and non-limiting.

It will be apparent to the reasonably skilled artisan which neurological disorders are suitable for treatment by the present methods based on cortical pathology and neuroanatomical connectivity. “Cortex” as used herein refers to the cerebral cortex. In some embodiments, the neurological disease or disorder is a CNS disease or disorder, e.g., Huntington's disease, Parkinson's disease, or Alzheimer's disease. In some embodiments, the neurological disease or disorder is a PNS disease or disorder, e.g., peripheral neuropathy.

According to one method, a viral vector is delivered to a central nervous system of a subject. The method includes providing a cannula assembly and a syringe combination including the cannula assembly and the syringe. The cannula assembly includes a cannula, polymeric support material and a hub. The cannula has a proximal end and a distal end. The polymeric support material substantially surrounds a portion of the cannula at or near the proximal end. The syringe includes a needle. The hub attaches the cannula assembly and the syringe. The polymeric support material is located between the cannula and the hub. The cannula, the polymeric support material and the hub are adhesively attached. The viral vector is provided and delivered to the central nervous system via the cannula assembly and the syringe combination.

According to another method, a neurological disorder is treated in a subject in need thereof. The method includes providing a cannula assembly and a syringe combination including the cannula assembly and the syringe. The cannula assembly includes a cannula, polymeric support material and a hub. The cannula has a proximal end and a distal end. The polymeric support material substantially surrounds a portion of the cannula at or near the proximal end. The syringe includes a needle. The hub attaches the cannula assembly and the syringe. The polymeric support material is located between the cannula and the hub. The cannula, the polymeric support material and the hub are adhesively attached. A viral vector is provided and is delivered to the central nervous system via the cannula assembly and the syringe combination. The neurological disorder is treated using the delivered viral vector to the central nervous system.

The neurological disorder in this method includes, but is not limited to, meningitis, encephalitis, multiple sclerosis (MS), stroke, brain tumors, epilepsy, Alzheimer's disease, AIDS-related dementia. Parkinson's disease or Huntington's disease.

In the present invention, the viral vector comprises therapeutic nucleic acid (e.g., DNA or RNA) within its genome. The rAAV can comprise any nucleic acid having therapeutic benefit. In some embodiments, the therapeutic nucleic acid is non-coding. For example, the therapeutic nucleic acid is non-coding RNA. Non-limiting examples of non-coding RNA are shRNA, siRNA, miRNA. In other embodiments, the therapeutic nucleic acid encodes therapeutic transgenes.

Non-limiting examples of therapeutic transgenes that can provide a therapeutic benefit for a disease or disorder of the CNS include CYP46A1 and HTT (For Huntington's), AADC and GDNF (for Parkinson's), GLB1 (for GM1), GDNF (for MSA), ASM (for Niemann-Pick), CYP46A1 (for Alzheimer's, ALS, MS and epilepsy), and UBE3A (for Angelman's). In some embodiments, the polypeptide-encoding transgene encodes an antibody or antigen-binding fragment thereof. Approaches for the treatment of CNS diseases or disorders can also: target metabolic pathways (e.g., CYP46A1 to clear protein-lipid rafts or protein aggregates for Huntington's, Parkinson's, ALS and Alzheimer's; similar approaches can also target synuclein and/or tau); use miRNA, shRNA and/or ribozyme meditated knockdown of undesirable mRNA transcripts (e.g., mHTT or HTT for Huntington's or ATS knockdown for Angelman's); use transgene expression for gene replacement (e.g., for restoring normal splicing by adding MBNL2 or SFRF6 in Huntington's, as well as more traditional gene replacement by expression of anti-synuclein antibodies, AADC, GDNF, or other transgenes). Diseases can include, among others, neurodegenerative diseases (Parkinson's, Huntington's, Alzheimer's, ALS, Multiple Sclerosis, epilepsy) and inborn mutations (AADC, Angelman, Newman-Pick, MPS, and others). Transgene-mediated gene editing is also contemplated, e.g., CRISPR or ARCUS or other gene editing technologies, including homologous recombination—this can be applied, for example, to Angelman disease.

### Example 1

### Delivery of Recombinant Viral Vectors Encoding AADC to Primate Brain

Recombinant AAV vector encoding human AADC (AAV2-hAADC) delivered to the putamen of rhesus monkeys as follows.

### Recombinant Vector Production

Recombinant AAV2 is generated by a triple transfection protocol (Matsushita et al. (1998) Gene Ther. 5(7) 938-45). Briefly, after expansion of cells from the HEK 293 working cell bank through a series of disposable culture ware in DMEM containing 10% fetal bovine serum and 2 mM glutamine, cells are co-transfected with three plasmids (pAAV-hAADC-2, pHLP 19 and pladeno5). The rAAV2-hAADC vector clone is the same as that described previously (Sanftner et al. (2004) Mol. Ther. 9(3): 403-9) Plasmids pHLP 19 and pladeno5 are described more fully at U.S. Pat. Nos. 5,139,941; 5,622,856; 6,001,650 and 6,004,797, the disclosures of which are hereby incorporated by reference in their entireties.

After an appropriate transfection time, the medium containing the transfection reagent is replaced with serum-free medium and the cells are incubated further to allow vector production Cells are harvested, concentrated by centrifugation, and lysed by a freeze/thaw method to release the AAV-hAADC-2 vector. After centrifugation to remove cellular debris, the lysate is treated with Benzonase®, calcium chloride, and precipitated with polyethylene glycol. Vector is purified by two cycles of isopycnic gradient ultracentrifugation in cesium chloride. AAV2-hAADC is concentrated, and diafiltered with sterile, buffered saline (PBS) containing 5% sorbitol. Poloxamer 1881™ (0.001%) is added, the material is sterile filtered (0.22 μm), and stored frozen at −70° C. Vector purity is assessed by SDS-PAGE. Purified rAAV2 vector to be used in this study show only VP1, VP2, and VP3 by silver staining of SDS-PAGE gels. Titer is determined by real-time Q-PCR analysis of vector genomes.

### Surgical Procedures

Stereotaxic coordinates (based on the anatomical structure of the putamen) are first identified in the Rhesus Monkeys. Two sites are targeted in each hemisphere with one site centered in the rostral putamen and a second in the caudal putamen. Adult rhesus monkeys (n=4) are immobilized with a mixture of ketamine (Ketaset®, 10 mg/kg, intramuscular injection) and Valium® (0.5 mg/kg, intravenous injection), intubated and prepared for surgery. Isotonic fluids are delivered intravenously at 2 mL-kg/hr. Anesthesia is induced with isoflurane (Aerane®, Omeda PPD, Inc., Liberty, N.J.) at 5% v/v, and then maintained at 1%-3% v/v for the duration of the surgery. The animal's head is placed in an MRI-compatible stereotaxic frame. Core temperature is maintained with a circulating water blanket while electrocardiogram, heart rate, oxygen saturation and body temperature are continuously monitored during the procedure. Burr-holes are made in the skull with a dental drill to expose areas of the dura just above the target sites. AAV2-hAADC is infused in two groups of monkeys-one group by: (1) cannula assembly of the invention (e.g., cannula assembly 10 shown in FIGS. 1A-1C); and the other group is infused by (2) a reference cannula assembly including a cannula and a needle that is press-fit into opposing ends of the rubber tubing. The rubber tubing in the reference cannula assembly attempts to bridge the space between the cannula and the needle. The reference cannula assembly lacks a hub and polymeric support material that is included in the cannula assemblies of the present invention.

Each monkey receives a total of 3×10¹¹ vg in 200 μL spread over four sites (50 μL per site with two sites per hemisphere). Infusion cannula assemblies are manually guided to the putamen in each brain hemisphere, and the animals receive bilateral infusions (i.e. sequential infusions to the rostral and caudal sites within both hemispheres) of AAV2-hAADC-(1.5×10¹² vg/mL) at infusion rates of 02 μL/min (10 min), 0.5 μL/min (10 min), 0.8 μL/min (10 min) and 1 μL/min (35 min) for the left hemisphere and a constant rate of 1 μL/min (50 min) for the right hemisphere. Approximately 10 minutes after infusion, the cannula assemblies are removed, the wound sites are closed, and the monkeys are monitored for recovery from anesthesia and then returned to its home cage for continuing observations.

A solution including gadolinium contrast agent is used along with an adeno-associated viral vector carrying an Aromatic L-amino acid decarboxylase gene (AAV2-AADC) to track the delivery of the viral vector solution. Visualization of gadolinium shows that the infused bolus tracks the movement of the cannula assembly tip and closely mimics the shape of the target anatomical structure. There, however, is expected to be a significant difference in intensity in gadolinium in the two groups of monkeys. The monkeys that receive the AAV2-hAADC-gadolinium using a cannula assembly of the present invention is expected to show significantly higher intensity of gadolinium compared to that in the reference group. Given equal volumes and equal amounts of viral vector and gadolinium being administered in both groups, the use of reference cannula assembly would be expected to clearly indicate leakage of the solution whereas the cannula assembly of the present invention is protected from leakage.

### Histology and Immunohistochemistry

For histological studies, animals are perfused via intracardiac saline infusion followed by 10% neutral buffered formalin (NBF). The brains are then removed and sliced in a brain mold into coronal blocks (8-10 mm). Harvested brain blocks are fixed by immersion in 10% NBF fixative. The tissue blocks are transferred 2-3 days after fixation into ascending concentrations of PBS/sucrose solution (10, 20 and 30%) over a 3 to 5 day period. Brains are frozen in a bath of isopentane, cooled on dry ice and cut serially into 40 μm thick coronal sections on a cryostat. Every tenth section is stained with Hematoxylin and Eosin (H&E) solutions (Richard Allen Scientific, Kalamazoo, Mich.) for histopathological analysis Immunohistochemistry is carried out on free-floating sections with a primary antibody specific for AADC (Chemicon, Temecula, Calif., 1:1,500). Sections are incubated in 3% hydrogen peroxide for 30 min to quench endogenous peroxidases. After blocking for non-specific binding with 10% normal goat serum, sections are incubated in primary antibody overnight at room temperature, then with a biotinylated anti-rabbit IgG antibody (Vector Laboratories, Burlingame, Calif., 1:300) with streptavidin-conjugated horseradish peroxidase (Vector Laboratories, 1:300) at room temperature, both for 1 h. The complex is visualized with 3-3′-diaminobenzidine (DAB, Vector Laboratories) and hydrogen peroxide. Sections are mounted on Superfrost Plus® slides (Brain Research Laboratories, Newton, Mass.), dried, dehydrated in ascending ethanol series, cleared in xylene, and mounted with Cytoseal-XYL (Richard-Allen Scientific, Kalamazoo, Mich.). Antenor-to-posterior distribution of hAADC immunostaining is determined by the formula (n×10×40 μm) where n is the number of sections with hAADC-positive cells, 40 μm is the thickness of the section, and every tenth section is examined. The volume of distribution is estimated in serial sections (every tenth), stained for AADC with the Optical Fractionator-Optical Dissector design-based stereology method under 63× magnification on a Zeiss microscope equipped with a video camera and Stereoinvestigator™ stereology software (Microbrightfield, Williston, Vt.). CEE is <5% for each group. Results are reported as mean±SD. Student's t-test was used to measure statistical significance.

### Real-Time Quantitative PCR

The vector AAV2-hAADC used in this study contains the human AADC target cDNA. The real-time Q-PCR primers and probe anneal to exons 2 and 3 of the AADC gene, spanning an intron not present in the vector sequence, thereby minimizing amplification of genomic DNA. Real-time Q-PCR is standardized with linearized plasmid DNA containing the vector insert and vector genomes are quantified as described previously (Sommer et al (2003) Mol. Ther. 7(1) 122-8).

Immunohistochemistry and Quantitation of hAADC Expression In-Vivo

Immunohistochemical analysis of hAADC expression is performed on each brain hemisphere at 5.5 weeks post-AAV-hAADC-2 infusion to determine if the vector distribution is different between the infusion by cannula assembly of the invention and the infusion by reference cannula assembly. All monkeys exhibit hAADC expression within the putamen. Serial sections are examined with brightfield microscopy for hAADC-positive cells. The volume of distribution and Anterior-Posterior (A-P) spread of hAADC transgene-positive cells are determined for all animals. The volume of distribution would be expected to be significantly different in two groups of monkeys: monkeys that are infused with the cannula assembly of the present invention would be expected to be significantly higher than the other group and this difference is attributed to the expected leakage of the viral vector solution in the reference cannula assembly compared to no leakage using the cannula assembly of the present invention.

In all animals, transgene expression is localized to the putamen. No hAADC expression is detected in cortical regions except in direct line with the infusion track. No difference in the number of AADC-positive cells or intensity of hAADC staining is seen in a comparison of the right and left hemispheres within each group of monkey. However, there is expected to be significantly less hAADC staining observed in the group where delivery is performed using reference cannula assembly, attributing to the observation with Gadolinium contrast agent at the time of delivering the viral vector. In other words, the reference cannula assembly is expected to show significant leakage as compared to the cannula assembly of the present invention that protects leakage. The observed less expression of hAADC in the group where reference cannula assembly used compared to the group having cannula assembly of the invention is therefore due to the significant less volume and hence less titer of viral vector being infused due to the leakage during delivery; all other experimental conditions between these two groups are otherwise the same.

Quantitative recovery of vector is evaluated through mock infusions using cannula assembly of the invention and reference cannula assembly. In both cases, AAV2-hAADC vector is diluted to 5-10¹¹ vg/mL (0.5×10⁹ vg/μL) After fill, both cannula assemblies are flushed with 500 μL of vector solution at 8 μL/min (62.5 min) Two sequential aliquots of 50 μL are collected from three sets of each cannula assembly at flow rates from 0.2 to 1.0 μL/min. Vector concentration in each sample is determined by real-time quantitative PCR (Q-PCR). Recovery for reference cannula assembly is only 49±15% after the extensive one-hour flush, whereas complete recovery of vector (101±3%) is observed for cannula assembly of the invention. This full vector recovery further confirms the superior protection from leakage in the cannula assembly of the invention.

Experiments described herein utilized animals with pre-existing NAb titers ranging from 1:1 to 1:100 to exclude neutralizing antibodies as a confounding variable, and these titers have no apparent impact on hAADC expression in putamen. Moreover, post-infusion titers rose only slightly after vector administration, thereby affirming well-targeted and minimally-disruptive gene delivery with the current device and infusion conditions. These results also suggest that repeat intrastriatal infusions of AAV2 may be feasible in human patients.

In summary, infusion of AAV2-hAADC to monkey putamen via cannula assembly of the present invention is expected to be protected from leakage and is well tolerated.

An embodiment of the cannula assembly of the present invention is tested to assess its ability to effectively deliver rAAV vector to primate brain, which may serve as a model for delivery of therapeutic rAAV vectors for treatment of CNS diseases and disorders in a human subject.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present invention have been described above, it should be understood that they have been presented by way of example only, and not limitation. Numerous changes to the disclosed embodiments can be made in accordance with the disclosure herein, without departing from the spirit or scope of the invention. Thus, the breadth and scope of the present invention should not be limited by any of the above described embodiments. Rather, the scope of the invention should be defined in accordance with the following claims and their equivalents.

1. A cannula assembly comprising:
a cannula having a proximal end and a distal end;
polymeric support material substantially surrounding a portion of the cannula at or near the proximal end; and
a hub configured to be attached to a syringe;
wherein the polymeric support material is located between the cannula and the hub,
wherein the cannula, the polymeric support material and the hub are non-detachable.

2. The cannula assembly of claim 1, wherein the cannula comprises glass fibers.

3. The cannula assembly of claim 1, wherein the polymeric support material is tapered.

4. The cannula assembly of claim 3, wherein the polymeric support material is tapered from the proximal end towards the distal end, in which the thickness of the polymeric support material is greater at the proximal end.

5. The cannula assembly of claim 1, wherein the polymeric support material comprises polytetrafluoroethylene (PTFE), polyamides, fluoropolymers, polyolefins, PVC (polyvinyl chlorides), polyimides, PEEK (polyetheretherketones), or combinations thereof.

6. (canceled)

7. The cannula assembly of claim 1, wherein the polymeric support material completely surrounds the cannula.

8-9. (canceled)

10. The cannula assembly of claim 1 further including a winged connection adapted to tighten the cannula assembly and the syringe.

11. The cannula assembly of claim 1, wherein the polymeric support material and the hub comprises transparent or translucent material.

12. The cannula assembly of claim 1, wherein the cannula, the polymeric support material, and the hub are non-detachably attached by a UV-sensitive adhesive.

13. The cannula assembly of claim 1, wherein the cannula, the polymeric support material, and the hub are non-detachably attached by way of shrink wrap.

14. (canceled)

15. A cannula assembly and a syringe combination comprising:
a cannula having a proximal end and a distal end, polymeric support material substantially surrounding a portion of the cannula at or near the proximal end, the polymeric support material located between the cannula and a hub, the polymeric support material and the hub being non-detachable; and
the syringe,
wherein the hub attaches the cannula and the syringe.

16. The combination of claim 15, wherein the polymeric support material is tapered.

17. The combination of claim 16, wherein the polymeric support material is tapered from the proximal end towards the distal end, in which the thickness of the polymeric support material is greater at the proximal end.

18-19. (canceled)

20. A method for forming a cannula assembly, the method comprising:
providing a cannula having a proximal end and a distal end, polymeric support material, and a hub, the polymeric support material and the hub comprising transparent or translucent material;
locating the polymeric support material to substantially surround a portion of the cannula at or near the proximal end, the polymeric support material located between the cannula and the hub; and
attaching the polymeric support material and the hub to the cannula to form a non-detachable, closed system.

21. The method of claim 20, wherein the polymeric support material is tapered.

22. The method of claim 21, wherein the polymeric support material is tapered from the proximal end towards the distal end, in which the thickness of the polymeric support material is greater at the proximal end.

23. (canceled)

24. The method of claim 20, wherein the attaching the polymeric support material and the hub to the cannula includes placing a UV adhesive on at least one of the cannula, the polymeric support material and the hub, and exposing the UV adhesive to UV light.

25. The method of claim 20, wherein attaching the polymeric support material and the hub to the cannula includes
shrink-wrapping the hub and the polymeric support material to the cannula.

26. A method of delivering a viral vector to a central nervous system of a subject, the method comprising:
providing the cannula assembly of claim 1;
providing a syringe containing a solution including the viral vector;
attaching the syringe to the hub of the cannula; and
administering the solution including the viral vector to the central nervous system of the subsect via the cannula assembly and the syringe.

27-30. (canceled)

31. The method of claim 26, wherein the central nervous system is brain tissue or a spinal cord.

32. (canceled)

33. The method of claim 26, wherein the subject has a neurological disorder.

34. The method of claim 33, wherein the neurological disorder is meningitis, encephalitis, multiple sclerosis (MS), stroke, brain tumors, epilepsy, Alzheimer's disease, AIDS-related dementia, Parkinson's disease or Huntington's disease.

35. The method of claim 34, wherein the neurological disorder is Alzheimer's disease, Parkinson's disease or Huntington's disease.

36. The cannula assembly of claim 1, wherein the cannula is configured to extend sufficiently into the hub so as to receive the syringe.

37. The cannula assembly of claim 1, wherein the cannula assembly is a non-detachable, closed sterile system.

38. A cannula assembly comprising:
a cannula having a proximal end and a distal end; and
a hub configured to be attached to a syringe,
wherein the cannula is configured to extend into the hub such that when a syringe is attached, the cannula is extended sufficiently to be received by the syringe so as to reduce the dead space relative to when the cannula is not extended sufficiently to be received by the syringe, and
wherein the cannula and the hub are non-detachable.

39. The cannula assembly of claim 38, further including a syringe to form a cannula assembly and syringe combination, the cannula extending into the hub and the syringe.

40. The cannula assembly of claim 38, wherein the cannula and the hub are attached by an adhesive.

41. The cannula assembly of claim 40, wherein the adhesive is a UV-sensitive adhesive.

42. The cannula assembly of claim 38, wherein the cannula and the hub are attached by shrink wrap.

43. The cannula assembly of claim 38, further including polymeric support material substantially surrounding a portion of the cannula at or near the proximal end, the polymeric support material being located between the cannula and the hub, the cannula, the polymeric support material and the hub being non-detachable.
